(19) United States
(12) Patent Application Publication
(10) Pub. No.: US 2012/0123192 A1
(43) Pub. Date: May 17, 2012

(54) BED SYSTEM AND A PORTABLE COVER DEVICE FOR A BED

(76) Inventors: Rajan Somsundaram, Berlin (DE); Birgit Liehr, Berlin (DE); Lisa Kroll, Berlin (DE)

(21) Appl. No.: 13/380,594

(22) PCT Filed: Jul. 19, 2010
(86) PCT No.: PCT/EP2010/004381

§ 371 Date: Jan. 25, 2012

(30) Foreign Application Priority Data
Jul. 17, 2009 (EP) 09009356.8

(51) Int. Cl. A61M 21/02; A61G 7/05; E04H 15/48

(52) U.S. Cl. 600/28; 135/143; 135/96; 600/27

(57) ABSTRACT

A bed system, comprising a bed (1), and a foldable cover device (6) mounted to the bed, wherein the foldable cover device (6) comprises a bearing structure (8) and a cover material (11) provided on the bearing structure (8), and wherein the foldable cover device (6) is movable between a folded position in which the cover device (6) is folded and an unfolded position in which the cover device (6) is unfolded, thereby at least partially covering a sleeping area (5) of the bed (1). (FIG. 1)

The invention refers to a bed system and a portable cover device for a bed.

### BACKGROUND OF THE INVENTION

There are challenges due to incidents that happen in hospitals: During peak time an old women was taken to the hospital with an acute, but not immediately life-threatening injury. Her daughter had informed the emergency staff about her mother's dementia. Consequently, to keep an eye on her during her waiting period she was put into a bed and placed in the hallway. However, after some time the patient left her bed and also the hospital. Nobody realized her absence for quite a while: Every staff member thought somebody else had taken her to an exam. A nurse removed the empty bed and the missing patient was missed even less. All the docs and nurses were extremely busy, anyways. Finally they realized that the patient had left the emergency department. With the help of the police she was found after a couple of hours at a nearby gas station. Not wearing a jacket in the middle of the winter, she was hypothermic, exhausted and of course very confused.

Dement patients are a growing problem in general. The older people get, the more they are diagnosed with Alzheimer's and other forms of dementia and the more they are admitted to the emergency department with some kind of acute condition.

The emergency department is a chaotic environment. The waiting room is unappealing and confusing. Waiting times are nontransparent and confusing. Many relatives communicate with their mobile phones, despite of prohibitions. Patients arrive via different ways at the emergency department. Either they are brought by an ambulance and put into a surgery room directly, or they are brought in by their relatives or come in themselves. Then they are either placed into a waiting bed or, if they are less severely injured, into a waiting room with seats. Usually their first contact is at the triage booth where a nurse categorizes patients according to their level of pain (triage). Then patients are either send to a bed directly or to the administration booth where their name and data are taken.

The layout of the emergency department is confusing: There are two parallel hallways with parallel room numbers. Guidance is almost non-existent and orientation is difficult. Despite opposite claims, there is lot of tension between different hierarchy levels and stakeholders: Doctors sometimes mistrust nurses, nurses mistrust their managing nurses, patients are angry, relatives feel left out, administrative staff feel under challenged. The preferred way of communication at the emergency department seems to be via notes and forms: There are little reminders and instructions everywhere and forms and guidelines for every imaginable situation. The main problem for staff members are time constraints and too less staff.

Dement patients are extremely stressful for the staff. Often they are aggressive or they run away. To tackle that problem they are placed in the hallway so that “everybody” can watch them. Sometimes they are also fixed with bed safety guard or sedated with drugs. Staff used work-arounds like post-it notes on the patient's back with their information or pictures of recurrent dement patients. However, there is no established routine to communicate dementia. Staff only talks about the acute condition of the patients. There is no dementia category at the triage form, either.

Dementia on the other hand, is a very complex and individual disease. It deeply affects the patient's personalities and their ability to life by themselves. It can be very different from patient to patient and at different phases of the disease. Gradually, dement patients do indeed forget nearly everything. However, obliviousness is not the only symptom. Patients loose their ability to correctly evaluate social situations, to locate themselves in space and time and to concentrate on “complex” tasks, such as getting dressed or talking on the phone. In addition, at an early stage many dementia patients still realize their condition, which often causes a depression. In a later stage, some patients feel the constant need to move, others are rather apathetic, some talk, laugh or mumble continuously, others fall silent or get aggressive. Patients aren't able to recognize their closest relatives or themselves anymore.

Concepts that accepts the patient's individual “quirks” work well: Circled hallways where they can wander around all day long, painted “bus stops” on the walls of the nursing home for them to wait for the bus. Many also like old children songs, old pictures and artifacts, like mechanical coffee grinders. Some like to dance and to dress up, some like smells and soft fabric to touch it. Some like to sleep with their mattress on the floor, others walk around at night. Although, they often don't recognize anybody, the nurses and relatives we talked to were sure that all patients still realized when somebody cared for them and paid attention to their needs.

In one nursing home the staff put chips under the patient's soles that activated an alarm as soon as the patient passed a light barrier at the building's exit. In another one, the door only opens with a code, which is openly displayed next to the touchpad—easy for visitors, nearly impossible for dementia patients.

### SUMMARY OF THE INVENTION

It is the object of the invention to provide an improved bed system.

The object is solved by a bed system according to independent claim 1. In addition, a portable cover device for a bed according to independent claim 12 is provided. Advantageous developments of the invention are disclosed in dependent claims.

According to one aspect of the invention, a bed system is provided, the system comprising a bed, and a foldable cover device mounted to the bed, wherein the foldable cover device comprises a bearing structure and a cover material provided on the bearing structure, and wherein the foldable cover device is movable between a closed position in which the cover device is folded and an unfolded position in which the cover device is unfolded, thereby at least partially covering a sleeping area of the bed.

According to another aspect of the invention, a portable cover device for a bed is provided, the device comprising a bearing structure and a cover material provided on the bearing structure, wherein the bearing structure is movable between a folded position in which the bearing structure is folded and an unfolded position in which the bearing structure is unfolded, thereby at least partially covering a sleeping area of the bed.

By means of the invention an individual treatment can be provided for a patient lying in the bed. Depending on the actual situation, by the foldable cover a shielding against the surrounding environment may be provided for the patient in the bed.

Preferably, the foldable cover device is mounted to a head end of the bed. In addition or as an alternative, the foldable cover device is mounted to other parts of the bed frame, for example a foot end and/or side rails. In one embodiment, the foldable cover device is detachably fixed to the bed.

In a preferred embodiment, the bed is a hospital bed. In general, the foldable cover device may be adapted for different kinds of beds. Preferably, adaptation is done by configuring mounting elements for different beds. The foldable cover device is configured for use with other hospital bed equipment, for example, a safety fence provided on one or both sides of the bed.

In a preferred embodiment of the invention, the bearing structure comprises a mechanical carrier structure.

In another preferred embodiment of the invention, the bearing structure is configured to be folded/unfolded by rotating bearing elements. In one embodiment, the bearing structure is provided with a securing mechanism configured to pivotally fix the bearing elements.

In still another preferred embodiment of the invention, the cover material is made of a washable material. In addition or alternatively, the cover material may be provided as at least one of a fire resistant material and a material which can be sterilized.

In still some other preferred embodiment of the invention, the foldable cover device comprises a stimulating device configured to output optical and/or acoustical stimulating signals to a patient. In one embodiment, the stimulating device comprises a display and/or a speaker device. In a preferred embodiment, a device configured to dispense a flavor to the patient is provided.

Preferably, the stimulating device comprises a display device provided on an inner side of the foldable cover device. The display device may comprise an active display for presenting video data to the patient. The active display, in one embodiment, comprises a foldable display made, example, of a thin layer OLED display (OLED—“Organic Light Emitting Diode”). In addition or alternatively, in a preferred embodiment a projection area is provided on the inside of the foldable cover device, preferably in a top roof area of the cover device. A video projector may be provided together with the foldable cover device to present video output to the patient on the projection area.

In a preferred embodiment of the invention, the foldable cover device comprises a sensor device configured to detect patient signals. Preferably, the sensor device configured to detect optical and/or acoustical patient signals. Optical signals may be detected by a camera device.

In another preferred embodiment of the invention, the foldable cover device comprises an information signaling device configured to optically and/or acoustically present patient information. The information signaling device may comprise an alarm system. In one embodiment, an information display device of the information signaling device is provided on an outer side of the foldable cover device. In addition or as an alternative, the information signaling device may be configured to send information signals to a central information system of the hospital.

In still another preferred embodiment of the invention, the information display device comprises a data exchange module configured to receive patient information data from and/or to send patient information data to a personal patient data carrier device. The data carrier device may be provided in a patient bracelet. In a preferred embodiment, data communication is implanted by wireless data communication. Electronic patient data received from the personal patient data carrier device may be used for operation-control of the functional elements of the cover device. Operation control is provided by a control device operable connected to one or all functional elements of the cover device. In one embodiment, the control device comprises a microprocessor and a memory. For example, control signals initiating stimulating signals to the patient in dependence on the data received from personal patient data carrier device are generated by the control device.

In still some other preferred embodiment of the invention, the foldable cover device comprises a control device configured to establish different operation modes for foldable cover device characterized by individual operation parameters. In a preferred embodiment, the control device is connected to at least one of the stimulating device, the sensor device, and the information signaling device.

Preferably, the bed is a mobile bed. In one embodiment, the foldable cover device is provided with at least one window allowing observation from the outside. Preferably, the window is a one-side window meaning there is no transparency looking at the window from the inner side where the patient is located.

### DESCRIPTION OF PREFERRED EMBODIMENTS OF THE INVENTION

Following the invention will be described in further detail, by way of example, with reference to different embodiments. In the figures:

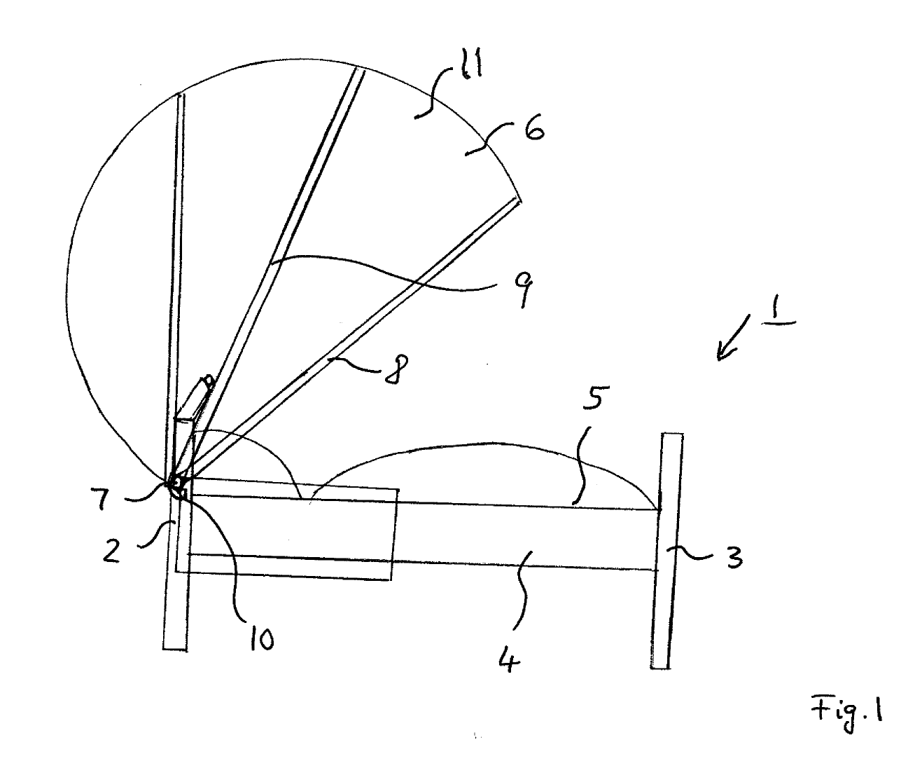

FIG. 1 shows a schematic representation of a bed system, wherein a mobile bed is provided with a foldable cover device in a first position which may be referred to as an unfolded position,

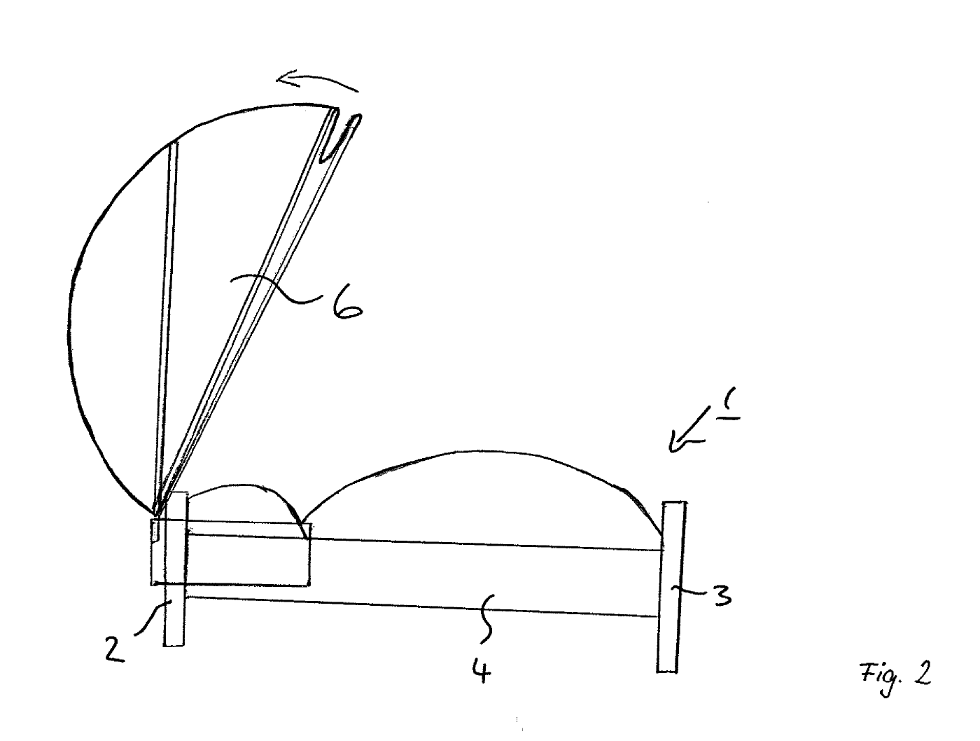

FIG. 2 shows a schematic representation of the bed system in FIG. 1, wherein the foldable cover device in a second position which may be referred to as an intermediate position, and

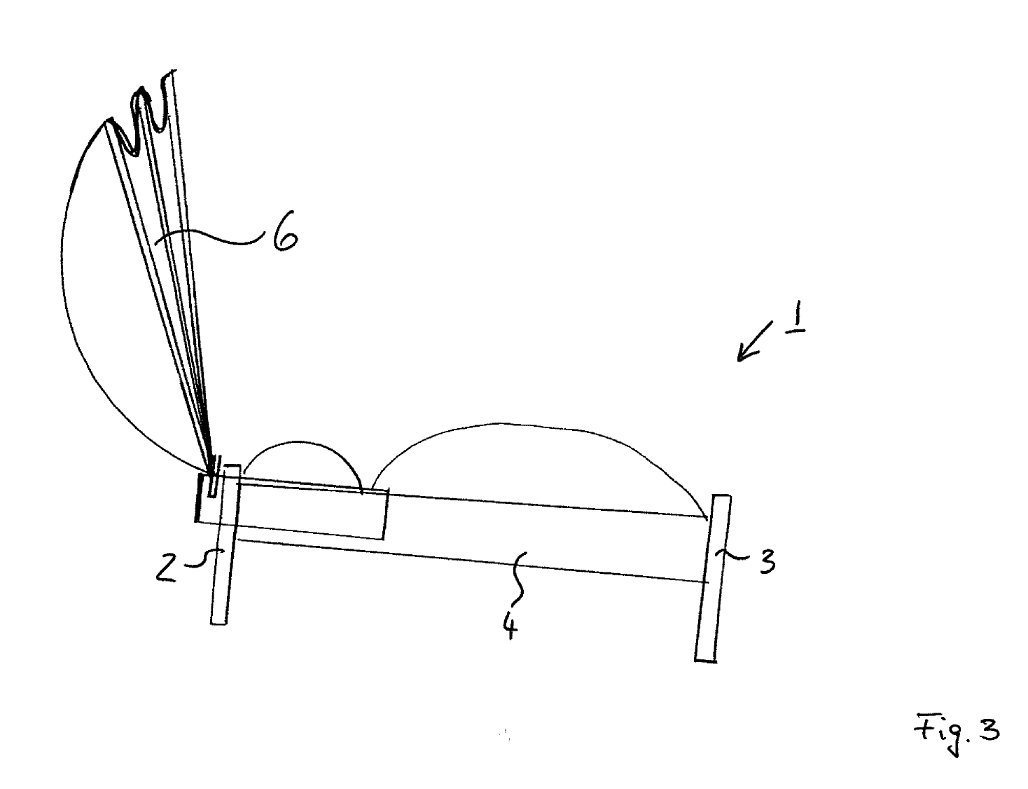

FIG. 3 shows a schematic representation of the bed system in FIG. 1, wherein the foldable cover device in a third position which may be referred to as a folded position.

In the course developing a new bed system, several needs were identified as follows.

Emergency department staff need to first recognize dement patients. Identification is not so much a problem of diagnosis. Most patients are brought in by their relatives or by an ambulance from their homes or nursing homes and their form of dementia are already known. However, that information is often lost at different points along the way. This leads to task one: The “d-info” has to arrive at the hospital together with the “d-patient”.

Secondly, once dement patients are identified the “dementia” information needs to stay connected to them. All too often, doctors and nurses only communicate the patient's acute injury. Thus, even if one staff member recognized and the dementia, the information is often lost at different points along the way. Sometimes staff used illegal work-arounds like labeling patients with post-its on their backs, stating their names and where they belonged. This leads to a task two: The “d-info” has to stay with the “d-patient” at all times.

The third need is the one that the hospital staff most clearly expressed themselves: Monitoring the dement patients. Some dement patients almost inevitably try to leave the emergency department, thereby endangering themselves and other people. However, during peak time, there is simply not enough staff to place somebody next to a dement patients to pay attention that they don't leave their bed. From this, a task three is derived: Staff needs to know the location of “d-patients” at all times.

The dement patients, on the other hand, are confused, nervous and anxious. They are faced with a total overload of information. They don't know where they are, what's going on or why they hurt. Strange, unknown people talk to them and try to explain them things they can't possibly understand. However, somehow everybody seems to expect them to understand. Imagine you are in a foreign country. You don't know why you are even there. You don't know where exactly you are or what time zone you are in. You don't understand the language and the signs, but the residents constantly instruct you and even expect important information and appropriate reactions from you. That's how a dement patient in an emergency department might feel. So they either react with embarrassment and try to cover up that nothing makes sense to them, or they express their anxiousness: They scream and moan, they get aggressive. Or they run away. Therefore, a task four may be considered: Minimize disorientation and anxiety for “d-patients”. Provide orientation and familiarity.

Also, the constraints at the emergency department have to be beard in mind: Lack of time, staff and resources, constant pressure, chaos and unforeseen, sudden disruptions are the norm not the exception. At the same time, there are numerous rules, regulations and routines that have to be strictly followed. Everything that obviously labels the dement patients inhibits their personal freedom and is legally forbidden. Also fixations that inhibit their freedom of movement are illegal. For the dement patient; on the other hand, the solution can't be too “technical”—it needs to contain technology, however it can't look like technology as they are not used to it. It can't be anything that would be attached loosely to their body as they would inevitably get afraid and remove it.

Starting from the above, a new bed system was created. FIG. 1 to 3 show a schematic representation of an embodiment of the bed system, wherein a mobile bed is provided with a foldable cover device in different positions which represent a first position which may be referred to as an unfolded position (FIG. 1), a second position which may be referred to as an intermediate position (FIG. 2), and a third position which may be referred to as a folded position (FIG. 3), respectively.

Referring to FIG. 1, a bed 1 is provided with a head end 2, a foot end 3, and side rails 4. Between the side rails 4 a sleeping area 5 is provided. In the embodiment depicted, the bed 1 is a mobile bed. A foldable cover 6 is mounted to the bed 1 at the head end 2 by fixing means 7. The foldable cover 6 may also be referred to as an extension, a hook or a top.

As shown in FIG. 1, the foldable cover 6 is provided with a mechanical bearing structure 8 made of bearing elements 9 pivotally mounted by a securing mechanism 10, and a cover material 11 providing an envelope. In FIG. 1, the foldable cover 6 is in a first position which may be referred to as an unfolded or expanded position.

FIG. 2 shows the foldable cover 6 in a second position which may be referred to as an intermediate or partially expanded position. Finally, in FIG. 3 the foldable cover 6 is in a third position which may also be referred to as a folded position. The foldable cover 6 is brought into the different positions by pivoting the bearing elements 9 together with the cover material 11.

Following, further aspects of the new bed system are described.

A mobile bed cover for dement patients is proposed which may also be referred to as a bedmobil. There is the foldable cover 6 made of washable, durable material that works analogous to a stroller convertible. If a dement patient is at the emergency department, emergency staff can quickly and with a few simple actions attach the foldable cover 6 to the patient's bed 1. Thereby the patient will be shielded from the chaotic surroundings at the emergency department. Inside the capsule provided by the foldable cover 6, different sensorial stimuli can be activated: Calming, visual images of example of a starry sky or nature. Also sounds like chirping or comforting music. Aromatherapy works well with some dementia patients, and they react well to haptic stimuli.

To fulfill the needs of emergency staff, the outside of the foldable cover 6 may flash in red as soon as the patient leaves the bed 1. Thereby the staff can immediately react and pay attention. In addition, the foldable cover 6 would work a as an identification device for the emergency staff: If they see the bed system they know that the patient placed under it has dementia.

In one embodiment, there is a combination of the bed system with a personalized bracelet (not shown) for dement patients. In addition, a process is used that already starts at the patient's permanent residence, i.e. their homes or the nursing homes. As soon as a person is diagnosed with dementia he or she will receive a permanent bracelet and a bed system as described above. Of course the patients and their relatives have to be informed about the device's details and functionality and have to consent to their use.

First, a dementia bracelet was provided: The patients would get such a bracelet either already at the ambulance or at the first contact booth at the hospital. In one embodiment, the bracelet contains a GPS device (GPS—Global Positioning System) to monitor and track the dement patients. The staff at the first contact booth would see the location of the dement patients on a map of the layout of the hospital. Optionally, a release buzzer for the emergency department doors may be provided with the patient bracelet to keep the patients inside. The bracelet would also contain the patient's names, their medical condition and information about their dementia. The patient bracelet could also monitor the patient's physical state and inform hospital staff about urgent needs of the patient.

The patient bracelet may be made of durable, washable material and it will be fixed to the patient's arm. It will contain a small and flat smart card that contains the patient's name, address, a contact person and some basic medical data. The patient bracelet is provided with a data storage device and a data communication module configured to receive and/or send data.

In the course of the disease, patients, nurses and family members can add relevant information to the card: Biographical dates that are important for the patient, pictures of his or her environment and of favorite items and close persons, reassuring music and the patient's needs and wishes. Also, in a later phase of the disease the patient will be categorized into one of four basic categories: Restless Runner, Lethargic Loner, Anxious Aggressor or Friendly Flirter.

The patient bracelet has different functions: Firstly, it works analogously to an electronically ankle manacle. As soon as patients try to leave their homes, a family member or nurse will be notified. As patients and/or their family members consented to the use of the bracelet it will not be an illegal restraint of their personal freedom. Also, the patients will be used to the bracelet and accept it as something familiar.

Secondly, the patient bracelet can be connected to the bed 1 via bluetooth technology. Thereby, every bed 1 can be individualized and used according to every patient's special needs. Every patient will have his or her own bed I at their place of residence. This will also increase the level of privacy in nursing homes, where the patients often live in shared bedrooms. They are able to fully retreat into their own world and thereby link their physical experiences and their sensations to their mental state. In addition, it will facilitate the care: Nurses and different caretakers are able to be informed about the patient's needs and wishes at all times.

If patients have an acute injury and need to be brought to the emergency department the information on their bracelets will naturally accompany them. Emergency staff will recognize dement patients with the bracelet. With a small scanning device utilizing bluetooth technology again, the nurse at the triage/first contact booth can put the relevant data into a computerized patient hospital management system. The data can now be seen whenever another doctor or nurse scrolls over the patient's name.

All dement patients will “automatically” be placed in a bed in combination with a bed. The bed 1 can again be individualized with the stored information on the patient bracelet: The patients have their individual home capsule away from home.

The patient's dementia category and status that is also stored on the bracelet, in combination with his or her acute condition will determine the alarm settings that also function as a combination of bed and bracelet. If the patient is not allowed to stand up, a nurse will be informed as soon as the patient tries to leave the bed. If the patient is categorized as Restless Runner and has a less severe injury, the alarm is set differently and will only be activated when the patient tries to leave the emergency department. Analogously to the alarm at the nursing home the staff at the triage/first contact booth will be informed and can immediately stop the patient or quickly alarm more staff to look for the patient.

The features disclosed in this specification, the claims, and the figures may be material for the realization of the invention in its various embodiments, taken in isolation or in various combinations thereof.

1. A bed system, comprising:
a bed (1), and
a foldable cover device (6) mounted to the bed,
wherein the foldable cover device (6) comprises a bearing structure (8) and a cover material (11) provided on the bearing structure (8), and wherein the foldable cover device (6) is movable between a folded position in which the cover device (6) is folded and an unfolded position in which the cover device (6) is unfolded, thereby at least partially covering a sleeping area (5) of the bed (1).

2. System according to claim 1, wherein the bearing structure comprises a mechanical bearing structure (8).

3. System according to claim 1, wherein the bearing structure (8) is configured to be folded/unfolded by rotating bearing elements (9).

4. System according to claim 1, wherein the cover material (11) is made of a washable material.

5. System according to claim 1, wherein the foldable cover device (6) comprises a stimulating device configured to output optical and/or acoustical stimulating signals to a patient.

6. System according to claim 1, wherein the stimulating device comprises a display device provided on an inner side of the foldable cover device.

7. System according to claim 1, wherein the foldable cover device (6) comprises a sensor device configured to detect patient signals.

8. System according to claim 1, wherein the foldable cover device (6) comprises an information signaling device configured to optically and/or acoustically present patient information.

9. System according to claim 8, wherein the information display device comprises a data exchange module configured to receive patient information data from and/or to send patient information data to a personal patient data carrier device.

10. System according to claim 1, wherein the foldable cover device (6) comprises a control device configured to establish different operation modes for foldable cover device characterized by individual operation parameters.

11. System according to claim 1, wherein the bed (1) is a mobile bed.

12. Portable cover device for a bed (1), comprising a bearing structure (8) and a cover material (11) provided on the bearing structure (8), wherein the bearing structure (8) is movable between a folded position in which bearing structure (8) is folded and an unfolded position in which bearing structure (8) is unfolded, thereby at least partially covering a sleeping area (5) of the bed (1).
